(19) United States
(12) Patent Application Publication
(10) Pub. No.: US 2004/0038354 A1
(43) Pub. Date: Feb. 26, 2004

(54) Bioproduct production during oxidisation of metal sulphide minerals by means of microorganisms

(76) Inventors: David William Dew, Paulshof (ZA); Chris Andre Du Plessis, Klofendal (ZA)

Correspondence Address: BRINKS HOFER GILSON & LIONE, P.O. BOX 10395, CHICAGO, IL, 60611, US

(21) Appl. No.: 10/444,541

(22) Filed: May 23, 2003

(30) Foreign Application Priority Data
Nov. 25, 2000 (ZA) 2000/5980

(51) Int. Cl. C12P 3/00

(52) U.S. Cl. 435/168

(57) ABSTRACT

A method of producing bioproducts which includes the steps of establishing an environment wherein microorganisms oxidise a slurry containing metal sulphide minerals, supplying a feed gas containing in excess of 21% oxygen by volume to the slurry, and extracting bioproducts from the slurry.

This application claims priority to and is a continuation of PCT WO 02/42504 published in English on May 30, 2002 and claims priority to ZA 2000/5980, the entire contents of both are incorporated herein by reference.

### BACKGROUND OF THE INVENTION

This invention relates generally to the production of bioproducts and particularly thermophilic bioproducts. More particularly the invention is concerned with creating an environment for bioprospecting.

As used herein the term ‘bioproducts’ includes microorganisms and their metabolites, e.g. proteins and, in particular, enzymes.

Unique microorganisms have been found at sites with unusual environmental conditions, for example at thermal springs at the Yellowstone National Park in America, in Iceland and New Zealand. Because of their characteristics these locations are generally protected and controlled by government bodies such as national parks boards. At these sites, microorganisms, mainly archaea, have adapted over millions of years to the prevailing harsh and extreme growth conditions. These archaea are unique mainly due to their capacity to withstand temperatures of between 70° C. to 90° C., acidity as low as a pH of 1, and high concentrations of dissolved metals, and due to their ability to utilise carbon dioxide as a carbon source.

The microorganisms not only survive but actively grow and flourish under these extreme circumstances. The fact that these microorganisms are active indicates the presence of enzymes that allow metabolic and physiological functioning under the prevailing conditions. Clearly these cells have a myriad of enzymes and enzyme systems, all of which have to be thermostable in order to function.

The potential value of enzymes and proteins contained in the microorganisms present in the extreme environments of the aforementioned kind has prompted governments and state organisations to impose strict control over bioprospecting for such microorganisms. The possible commercial value of thermostable enzymes is apparent from the numerous requests to bioprospect on these naturally occurring sites for potentially valuable bioproducts. One example of the way in which these sites can be exploited is the isolation of the enzyme Taq YT-1 from an archaea called Thermus aquaticus, which was collected from the Yellowstone National Park. This enzyme is vital to the Polymerase Chain Reaction process, which has since become the cornerstone of modern medical diagnostic work, resulting in substantial sales of the enzymes. On the other hand, the possibility of identifying and subsequently commercially exploiting enzymes derived from such sites is problematic due to the fact that such sites are generally state-owned and, to an ever-increasing extent, the microorganisms occurring at such sites are regarded as natural resources which must be protected. Most such sites, for example, are subject to the provisions of the Convention on Biological Diversity (1992). This controls access to the sites, requiring prior informed consent from national governments for sample collection, and equitable sharing of the benefits arising from such access.

### SUMMARY OF THE INVENTION

According to a first aspect of the invention bioleaching processes for the extraction of metals are adapted to the production of bioproducts.

The extraction of metals from ores using biological agents (microorganisms), particularly thermophilic bacteria, is a useful technique, often known as bioleaching. Older processes for extracting metals from ores employ strong heat and toxic chemicals. Using bioleaching, metals may be extracted under more environmentally-friendly conditions, and from ores of lower metal content. For example, copper sulphide ore may be digested with the bacterium Acidithiobacillus ferrooxidans in the presence of oxygen. This converts the sulphide ore to water-soluble sulphate, and the resulting copper sulphate solution is separated from the slurry. Metallic copper is then recovered from the solution by electrowinning. The solid constituents of the slurry are waste materials, which may be disposed of, for example, on slagheaps.

The invention provides a method for the production of bioproducts, which comprises digesting metal ore with microorganisms in a reactor, separating metallic products and residues from a reaction mixture, and recovering bioproducts from the reaction mixture.

The metallic products and residues are generally metal salts in solution, or undigested ore.

The method may include the steps of:

(a) establishing an environment, under controlled conditions, wherein microorganisms are used to oxidise a slurry containing metal sulphide minerals, and

(b) separating and recovering bioproducts from the slurry.

The environment may be established in at least one reactor vessel.

The bioproducts which are recovered may be microorganisms or their metabolites, or both in admixture.

According to circumstances, the recovered bioproducts may be of known or unknown constitution. In one form of the invention the method includes the step of recovering economic quantities of bioproducts for further industrial use.

Thus useful bioproducts may be recovered directly from the reaction slurry. Such bioproducts are typically present in the slurry in the solid phase, and may be extracted from the slurry by various methods after separation of the solid from the aqueous phase of the slurry. These methods may include washing or extraction with various solvents, centrifugation and the like.

The uses to which the bioproducts are put will vary according to their nature. One such use is to be recycled as bioleaching agents.

An additional feature of the invention is to use recovered bioproducts, in particular enzymes, for further mineral extraction processing. Enzymes in the bioproducts obtained by the application of the invention can enable such further mineral extraction processing to be carried out under more extreme conditions (e.g. at a higher temperature or in the presence of heavy metal concentration), leading to faster or more efficient processing. The enzymes generally resist more extreme conditions than the organisms which produce them.

In a different form of the invention the method includes the step of screening recovered bioproducts for desired properties. This approach provides access to bioproducts of interesting and uncommon properties: for example, enzymes which are stable at relatively high temperatures and in harsh environments. Such enzymes, and organisms producing them, are widely sought for various uses, but access to them is not straightforward.

This aspect of the invention therefore provides a useful alternative source of bioproducts resistant to extreme environmental conditions such as heat, acidity and metal concentrations.

The method of the invention may include the step of supplying the said slurry to the reactor vessel in batch form, but preferably the slurry is supplied in a continuous stream.

The sulphide minerals may include pyrite, arsenopyrite, pentlandite, enargite, chalcopyrite, sphalerite, cinnabar, covelite, and bornite.

Due to the oxidation of the metal sulphides a high concentration of dissolved metals may prevail in the slurry. These metals may include nickel, iron, copper, zinc and arsenates. The presence of compounds of these metals, in solution, could inhibit growth of the microorganisms and under such conditions the microorganisms would have to employ strategies to survive and to prevent cell inhibition. These strategies would necessarily involve metabolic and physiological functions which would be facilitated by the activity of specific enzymes and proteins.

It does however fall in the scope of the invention to add further or additional dissolved metals in a soluble form to the reactor vessel.

The method may include the step of introducing small amounts of organic compounds such as glucose into the said environment in order to cater, and to provide greater selectivity, for specific microorganisms with a growth requirement which is met by these organic compounds.

Alternatively or additionally inorganic nutrients may be supplied to the slurry to achieve any appropriate and desired selectivity factor and, in particular, to optimise the growth of the microorganisms in the reactor or reactors in which the method is implemented.

The inorganic nutrient supplements may include nitrogen, phosphorus and potassium. These may be added in any appropriate form and in any suitable concentration.

By way of example inorganic nutrients may be added as follows, in the indicated concentrations, and in the indicated compositions:

Typical nutrient concentrations
added as a function of concentrate solids:

| | | |
|---|---|---|
| Nitrogen | 1.50 kg ton⁻¹ | added as (NH₄)₂SO₄ |
| Phosphorus | 0.60 kg ton⁻¹ | added as KH₂PO₄ |
| Potassium | 0.75 kg ton⁻¹ | added as KH₂PO₄ and K₂SO₄ |

Although micro-nutrients may be added to the slurry this will not normally be required due to the fact that compounds of the micro-nutrients generally occur in trace element quantities in the mineral concentrates.

The method may include the step of applying specific mutagenic pressure to the microorganisms in the reactor vessel in order to promote adaptation of the microorganisms to particular conditions. The nature of the mutagenic pressure may vary according to requirement and for example may include the exposure of some cells to ultraviolet or gamma radiation, and the introduction of appropriate concentrations of mutagenic chemicals into the slurry.

The method may include the step of controlling the mineral solids concentration of the slurry in the reactor vessel. This step may be used to facilitate the selection of microorganisms with associated physiological functions capable of withstanding the high shear forces associated with a high solids loading.

The reactor vessel may include an impeller for agitating the slurry and the method may include the step of controlling the impeller speed, or the degree of agitation, in order to select for microorganisms with associated physiological functions capable of withstanding high shear forces associated with high impeller tip speeds.

The method may include the step of controlling the hydraulic retention time of the reactor vessel. If the slurry is fed substantially continuously to the reactor vessel the microorganism growth rate is controlled by, and is substantially equal to, the reactor dilution rate (which is the reciprocal of the hydraulic retention time). This feature can be used to select for microorganisms with a specific cell growth rate under the prevailing reactor conditions.

The method may include the step of sparging the slurry in the reactor vessel with air which may be supplemented with additional carbon dioxide or oxygen thereby resulting in stable and elevated dissolved carbon dioxide or oxygen concentrations.

In order to establish a dissolved oxygen concentration level in the slurry at an elevated value, in an effective and efficient manner, the method may include the step of feeding an oxygen-enriched gas to the slurry.

As used herein the expression “oxygen enriched gas” is intended to include a gas, e.g., air, which contains in excess of 21% oxygen by volume, which is greater than the oxygen content of air. The expression “pure oxygen” is intended to include an oxygen-enriched gas which contains in excess of 85% oxygen by volume.

Preferably the feed gas which is supplied to the slurry contains in excess of 85% oxygen by volume i.e. is substantially pure oxygen.

The method may include the step of maintaining the dissolved oxygen concentration in the slurry within a desired range which may be determined by the operating conditions and the type of microorganisms used for leaching. The elevation of the dissolved oxygen concentration in the slurry in the reactor vessel contributes to the uniqueness of the microorganism growth environment as no similar hyper-oxygenated environments occur naturally. The unique elevated dissolved oxygen concentrations may give rise to the selection and adaptation of microorganisms with growth functions and specific novel enzymes and enzyme systems, or initial higher activity functions of such enzyme systems.

The dissolved oxygen concentration may be maintained in the range of from 0.2×10⁻³ kg/m³ to 10×10⁻³ kg/m³, with the value being chosen to achieve optimal growth of the microorganisms.

The method may include the steps of determining the dissolved oxygen concentration in the slurry and, in response thereto, of controlling at least one of the following: the oxygen content of the feed gas, the rate of supply of the feed gas to the slurry, and the rate of feed of the slurry to the reactor vessel.

The dissolved oxygen concentration in the slurry may be determined in any appropriate way, e.g. by one or more of the following: by direct measurement of the dissolved oxygen concentration in the slurry, by measurement of the oxygen content in gas above the slurry, and indirectly by measurement of the oxygen content in off-gas from the slurry, taking into account the rate of oxygen supply, whether in gas enriched or pure form, to the slurry, and other relevant factors.

The method may include the step of controlling the carbon content of the slurry. This may be achieved by one or more of the following: the addition of carbon dioxide gas to the slurry, and the addition of other carbonaceous material to the slurry.

The method may extend to the step of controlling the carbon dioxide content of the feed gas to the slurry in the range of from 0.5% to 5% by volume. A suitable figure is of the order of 1% to 1.5% by volume. The level of the carbon dioxide is chosen to maintain high rates of microorganism growth and sulphide mineral oxidation.

The said environment is preferably maintained at an elevated temperature. Generally the bioleaching rate and the microorganism growth rate increase with an increase in reactor operating temperature. Clearly the microorganisms which are used for bioleaching are determined by the operating temperature. Thus the said environment may be maintained at a temperature of up to 100° C. or more and preferably is maintained at value which lies in a range of from 60° C. to 85° C.

The method may include the step of initiating microorganism growth in the reactor by introducing a suitable microorganism inoculum into the slurry. The inoculum may be obtained from any appropriate source such as, for example, liquid or solid samples from sulphur-containing coal dumps which may be high temperature dumps, sulphur-containing volcanic thermal areas including such areas which are in contact with sea water, or which are submerged at substantial depths in sea water, and sulphur-containing inland thermal hot springs.

In one form of the invention the environment is maintained at a temperature of up to 45° C. and the slurry is oxidised using mesophile microorganisms. These microorganisms may, for example, be selected from the following genus groups Acidithiobacillus (formerly Thiobacillus); Leptospirillum; Ferromicrobium; and Acidiphilium.

In order to operate at this temperature the said microorganisms may, for example, be selected from the following species Acidithiobacillus caldus (Thiobacillus caldus); Acidithiobacillus thiooxidans (Thiobacillus thiooxidans); Acidithiobacillus ferrooxidans (Thiobacillus ferrooxidans); Acidithiobacillus acidophilus (Thiobacillus acidophilus); Thiobacillus prosperus; Leptospirillum ferrooxidans; Ferromicrobium acidophilus; and Acidiphilium cryptum.

In a variation of the invention the environment is maintained at a temperature of from 45° C. to 60° C. and the slurry is oxidised using moderate thermophile microorganisms. These may, for example, be selected from the following genus groups Acidithiobacillus (formerly Thiobacillus); Acidimicrobium; Sulfobacillus; Ferroplasma (Ferriplasma); and Alicyclobacillus.

Suitable moderate thermophile microorganisms may, for example, be selected from the following species Acidithiobacillus caldus (formerly Thiobacillus caldus); Acidimicrobium ferrooxidans; Sulfobacillus acidophilus; Sulfobacillus disulfidooxidans; Sulfobacillus thermosulfidooxidans; Ferroplasma acidarmanus; Thermoplasma acidophilum; and Alicyclobacillus acidocaldrius.

In a preferred embodiment the environment is maintained at a temperature in the range of from 60° C. to 85° C. and the slurry is oxidised using thermophilic microorganisms. These may, for example, be selected from the following genus groups Acidothermus; Sulfolobus; Metallosphaera; Acidianus; Ferroplasma (Ferriplasma); Thermoplasma; and Picrophilus.

Suitable thermophilic microorganisms may, for example, be selected from the following species Sulfolobus metallicus; Sulfolobus acidocaldarius; Sulfolobus thermosulfidooxidans; Acidianus infernus; Metallosphaera sedula; Ferroplasma acidarmanus; Thermoplasma acidophilum; Thermoplasma volcanium; and Picrophilus oshimae.

According to a different aspect of the invention there is provided a method of producing bioproducts which includes the steps of:

(a) culturing microorganisms which are capable of oxidising mineral sulphides contained in a slurry at a temperature in excess of 40° C.,

(b) controlling the dissolved oxygen concentration in the slurry within a predetermined range, and

(c) extracting bioproducts from the slurry.

The said predetermined range may be from 0.2×10⁻³ kg/m³ to 10×10⁻³ kg/m³ with the concentration value being determined to optimise the growth rate of the microorganisms.

The said dissolved oxygen concentration may be controlled by controlling the supply of oxygen to the slurry.

The oxygen may be supplied to the slurry in the form of oxygen-enriched gas or substantially pure oxygen.

The said operating temperature is preferably in excess of 60° C. and may lie in the range of from to 60° C. to 85° C.

Other physical parameters, relating to the slurry, which may be varied in a controlled manner include the following: the dissolved carbon dioxide concentration in the slurry, the temperature of the slurry, the pH of the slurry, the supply of organic nutrients to the slurry, the exposure of the slurry to mutagenic factors, the mineral solids concentration in the slurry, the hydraulic retention time of the slurry in a reactor vessel, and the imposition of the high shear forces, which may be mechanically induced, on the slurry.

The invention also extends to a method of producing bioproducts which includes the steps of:

(a) establishing an environment wherein microorganisms oxidise a slurry containing metal sulphide minerals,

(b) supplying a feed gas containing in excess of 21% oxygen by volume to the slurry, and

(c) extracting bioproducts from the slurry.

The feed gas preferably contains in excess of 85% oxygen by volume.

The method may be carried out at a temperature in excess of 60° C. and the temperature may for example lie in the range of from 60° C. to 85° C.

Bioproducts may be extracted from the slurry or from samples which are derived from the slurry. These products may be used directly in subsequent manufacturing and commercialisation activities: or may be screened to discover components of interest.

The screening of bioproducts may be carried out using techniques which are known in the art. The bioproducts are of two general types: microorganisms and proteins (enzymes). These are initially obtained in admixture and it is preferred to separate such mixtures before screening them. Initial testing may concentrate on properties of enzymes, by procedures which may include one or more of the following: collection of small or large microbial samples; enzyme extraction and purification; screening of large numbers of purified and semi-purified enzymes for activity under particular conditions. The methods of screening for enzymes of interest are known to the art, and will depend on the properties sought.

Enzymes likely to be obtainable by the method of the invention have a wide range of useful properties and applications, some of which are considered in more detail hereinafter. Discovery of an enzyme of interest will provoke investigation of the organism producing it. This may be followed by isolation of the microorganism and its use to produce the enzyme of interest in commercial quantities under controlled conditions. The DNA of mircroorganisms in the bioproducts (particularly those associated with enzymes of interest) may be investigated, using techniques such as: creation of gene expression libraries; creation of libraries of multi-gene pathways responsible for the production of small molecules; screening of large numbers of genes and their variants. Such techniques, which may involve storage and manipulation of information generated from screening activities, can lead to discovery of the gene sequence (or gene pathway) leading to the production of an enzyme of interest. This may enable, in suitable cases, insertion of a selected recombinant gene or pathway into host organisms, enabling more efficient manufacture of the desired enzyme (gene expression product) or variants of it improved by genetic recombination.

Enzymes may be separated from the slurry using various centrifugal, filtration and precipitation processes. These processes are however generally known in the art and for this reason are not described herein in detail.

### BRIEF DESCRIPTION OF THE DRAWINGS

The invention is further described by way of example with reference to the accompanying drawings, in which

FIG. 1 illustrates in block diagram form the creation of an environment, under controlled conditions, for carrying out the method of the invention; and

FIG. 2 shows in block diagram form bioproduct separation stages of FIG. 1 in greater detail.

### DESCRIPTION OF PREFERRED EMBODIMENT

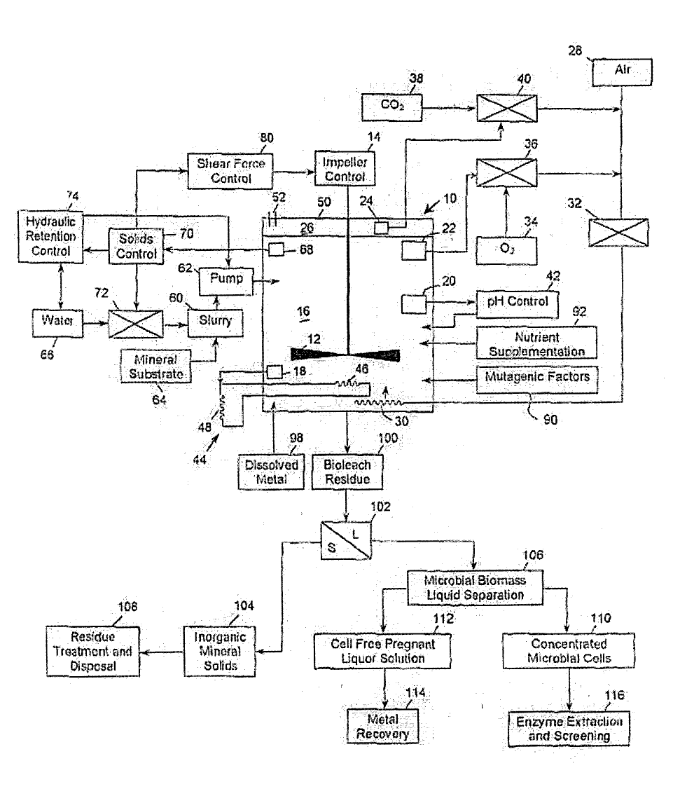

FIG. 1 of the accompanying drawings illustrates a reactor vessel 10, which may be one of a plurality of similar vessels arranged in series, in which a controlled environment is established, in accordance with the principles of the invention.

The vessel 10 includes an impeller 12 which is driven by means of a motor and gearbox assembly, not shown, the operation of which is regulated by an impeller control unit 14.

In use the vessel 10 contains a copper sulphide mineral slurry 16 and the impeller 12 is immersed in the slurry and is used for mixing the slurry in a manner which is known in the art.

A plurality of probes monitors physical parameters of the slurry in the vessel. These probes include a temperature probe 18, a pH sensor 20, a probe 22 which measures the dissolved oxygen concentration in the slurry, and a probe 24 which is exposed to the gas 26 in the vessel 10, above the slurry, and which measures the carbon dioxide content in this gas.

Air from a source 28 is fed to a sparging system 30 in the slurry in a lower region of the vessel. The rate at which air is introduced into the slurry is controlled by means of a valve 32. The probe 22 controls the addition of oxygen from a source 34 through a valve 36 to the air stream to the sparging system. Similarly the supply of carbon dioxide from a source 38, through a valve 40, is controlled by the probe 24.

The pH level of the slurry is monitored by the sensor 20 and may be lowered or increased, according to requirement, by the addition of a suitable acid or alkaline medium 42. Generally, as is known in the art, the pH of the slurry may be adjusted by the addition of 10N H₂SO₄ as required.

The temperature in the slurry is measured by the probe 18 and is controlled in any appropriate way using techniques which are known in the art. In one example the vessel 10 is insulated and heating takes place by means of energy which is released by the oxidation of sulphides. The temperature of the slurry 16 is regulated using a cooling system 44 which includes a plurality of heat exchanger coils 46 which are immersed in the slurry and which are connected to an external heat exchanger 48.

The vessel 10 may be substantially sealed by means of a lid 50. Small vents 52 are provided to allow for the escape of off-gas. Alternatively, according to requirement, the vessel 10 may be open to atmosphere.

The microorganisms which are chosen for the leaching process will determine the leaching temperature, and vice versa. In order to create conditions which are conducive for the growth of unusual microorganisms it is preferred to operate at a temperature in excess of 60° C., for example in the range of 60° C. to 85° C. In this range thermophilic microorganisms, in any appropriate combination, may be employed for the oxidising step. In the range of from 45° C. to 60° C., on the other hand, moderate thermophiles are employed while at temperatures below 45° C. mesophiles are used. These microorganisms may, for example, be chosen from those referred to hereinbefore.

Slurry is fed from a source 60 to the vessel at a rate which is controlled by means of a pump 62. The slurry is derived from at least one mineral substrate 64 which is chosen to produce a particular operating condition in the vessel 10. Water from a source 66 is added to the substrate in order to vary the mineral solids concentration in the slurry in the vessel. A probe 68 measures the solids concentration in the slurry and through the medium of a solids control unit 70 regulates the supply of water from the source 66 through a valve 72 to the slurry.

The hydraulic retention time of the reactor vessel is also controlled by regulating the rate at which slurry is supplied to the vessel. This aspect is controlled by a unit 74 which is responsive to the solids concentration in, and water content of, the slurry, and which acts on the pump 62. When the vessel is operated in a continuous culture mode the microbial growth rate is controlled by, and is substantially equal to, the reactor vessel dilution rate which is equal to the reciprocal of the hydraulic retention time.

It follows that the slurry composition is variable to control the mineral solids concentration in the reactor vessel and the slurry feed rate to the vessel is variable to regulate the hydraulic retention time of the vessel.

The impeller control unit 14 is capable of driving the impeller to establish high impeller tip speeds in order to generate high shear forces in the slurry. The shear forces additionally depend on the solids content in the slurry and accordingly a shear force control unit 80 is employed, which is responsive to the solids content in the slurry, measured by means of the probe 68, to regulate the operation of the impeller control unit 14.

Specific mutagenic factors or pressures may be applied to the slurry to facilitate or expedite adaptation of the microbial cells to the prevailing reactor conditions. This aspect is indicated schematically by means of a block 90 which represents a mechanism for exposing at least a small portion of the slurry to ultraviolet or gamma radiation. Alternatively or additionally the block 90 may represent a source of mutagenic chemicals which are introduced at a controlled dosage rate into the slurry.

Another variable in the reactor conditions is indicated by a block 92 which represents the introduction of small amounts of organic compounds, such as glucose, into the slurry. These compounds cater for greater selectivity of specific cells which require the organic supplementation for expedited growth.

Alternatively or additionally the block 92 represents the addition of inorganic nutrients to the slurry 16, in a manner which is known in the art, to optimise microbial growth. Typical nutrient concentrations which are added as a function of concentrate solids are as follows:

| | | |
|---|---|---|
| Nitrogen | 1.50 kg ton⁻¹ | added as (NH₄)₂SO₄ |
| Phosphorus | 0.60 kg ton⁻¹ | added as KH₂PO₄ |
| Potassium | 0.75 kg ton⁻¹ | added as KH₂PO_{4 and K}₂SO₄ |

The typical nutrient concentrations which are required in solution (as per DSMZ-Deutsche Sammiung von Mikroorganismen und Zellkulturen GmbH, Braunschweig, Germany, Culture Medium Number 88) are as follows:

Macro-Nutrients

| | |
|---|---|
| (NH₄)₂SO₄ | 1.30 gL⁻¹ |
| KN₂PO₄ | 0.28 gL⁻¹ |
| MgSO₄. 7 H₂O | 0.25 gL⁻¹ |

Micro-nutrients

| | |
|---|---|
| CaCl₂.₂H₂O | 0.07 gL⁻¹ |
| FeCL₃.6 H₂O | 0.02 gL⁻¹ |
| MnCl₂.4 H₂O | 1.80 gL⁻¹ |
| Na₂B₄O₇.10 H₂O | 4.50 gL⁻¹ |
| ZnSO₄.7 H₂O | 0.22 gL⁻¹ |
| CuCl₂.2 H₂O | 0.05 gL⁻¹ |
| Na₂MoO₄.2 H₂O | 0.03 gL⁻¹ |
| VOSO₄.2 H₂O | 0.03 gL⁻¹ |
| CoSO₄ | 0.01 gL⁻¹ |

It is apparent therefore that it is possible, within the reactor vessel 10, to establish and maintain a unique environment. Physical conditions which can be varied or controlled include the following:

(a) the creation of high concentrations of dissolved metals such as nickel, iron, copper and zinc, and arsenates, in the slurry. This aspect is determined by the specific feed source minerals in the substrate 14. Microbial oxidation of the metal sulphide leads to the release of the associated metal. It is possible nonetheless to add further or additional concentrations of dissolved metals directly to the slurry to induce specific reactions, as is indicated by a block 98;

(b) organic nutrient supplements may be added to the slurry to cater for the specific requirements of microbial cells;

(c) mutagenic pressures in radiation or chemical form may be applied to the slurry;

(d) a variation in the mineral solids concentration in the slurry;

(e) high shear forces can be imposed on the slurry. This enables selection of microbial cultures with associated physiological functions capable of withstanding the high shear forces;

(f) the hydraulic retention time of the reactor is controllable to select for organisms with a specific cell growth rate;

(g) the dissolved oxygen concentration in the slurry may be maintained within a desired range. It has been found that by sparging the slurry with an oxygen-enriched gas it is possible to increase the dissolved oxygen concentration in the slurry to a level which is substantially greater than what is achieved using only air, and in a far more effective manner. This contributes to the creation of an unique microbial growth environment as no similar hyper-oxygenated environments occur naturally. The high dissolved oxygen concentration level gives rise to the selection and adaptation of microorganisms with growth functions and specific enzymes and enzyme systems which are not duplicated in naturally occurring systems;

(h) similarly, by monitoring the carbon dioxide content in the gas 26 above the slurry, and then controlling the addition of carbon dioxide to the air stream 28, the carbon content of the slurry may be adjusted. Nonetheless it falls within the scope of the invention to add other carbonaceous material to the slurry;

(i) a highly important parameter is the elevated temperature at which the slurry is maintained. By suitable control of the cooling system 44 the slurry may be maintained at a temperature in the range of from 60° C. to 85° C.; and

(j) yet another variable is the introduction of a suitable microbial inoculum into the slurry. The inoculum may be derived from any appropriate source such as a sulphur-containing thermal spring, from mine tailings, e.g. a coal dump or from a submerged thermal site at sea.

The microbial oxidation of the slurry containing metal sulphide minerals at elevated temperatures and at the unique conditions which are imposed by suitable control of the variable factors implies that unique conditions exist for the growth of thermostable microorganisms. These microorganisms are then subjected to bioprospecting for the identification of novel bioproducts such as enzymes and genes.

The oxidising process produces a bioleach residue 100 which is then subjected to a solid/liquid separation step 102 producing inorganic mineral solids 104 and a microbial biomass liquid 106.

The solids 104 are subjected to residue treatment before disposal (108). The solution 106 is subjected to a process for extracting concentrated microbial cells 110 and a cell free pregnant liquor solution 112 from which metal is recovered (114). The cells 110 are screened (116) for enzymes.

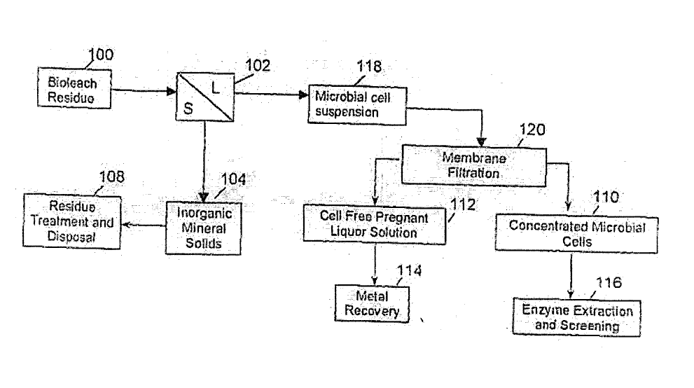

FIG. 2 shows in more detail a specific method for the separation of biomass (microbial cells) from minerals, solid particles and liquid solution by the separation of minerals solids from the bioleach slurry and the separation, and concentration, of the microbial cell biomass from the rest of the bioleach liquor.

The bioleach residue 100, in the form of a slurry, reports to a gravitational settler or clarifier 102 in which the larger and denser mineral particles settle out of suspension and collect (concentrate) at the bottom of the clarifier. This material is then discarded as indicated (stages 104 and 108). The gravitational settling of the mineral particles results in a supernatant 118 which is relatively free of mineral particles and in which a significant portion of the microbial cells remain in suspension. This step may alternatively make use of a centrifuge, instead of a gravitational settler, particularly for a situation in which the slurry contains a significant portion of very fine mineral particles which cannot efficiently be removed from suspension using a gravitational settler.

In a second phase of this process, the microbial cell-containing supernatant 118 from the gravitational settler (or centrifuge) 102 reports to a membrane filtration process 120. A suitable reactor is divided by a ceramic membrane which serves to retain the microbial cells within the reactor while the pregnant liquor solution (bioleach solution) 112 is passed through the membrane surface, to collection in a suitable vessel and subsequent metal recovery 114. This action results in a high microbial cell concentration solution 110 which facilitates the downstream use of microbial cell biomass for enzyme extraction and screening (116), optionally followed by recycling to a bioleaching reactor.

Instead of using the membrane filtration process 120, cell separation and concentration from the microbial cell suspension could, alternatively, be achieved using a centrifuge operating for example, at speeds generating a force of 7000G or more.

The screening 116 may be carried out on the concentrated cell solution 110 using suitable techniques such as proteonics and genonics, and those referred to by the proprietary names of phenomics™, GSSM™ (gene site saturation mutagenesis), GeneReassembly™, DirectEvolution®), and L-Shuffling™. Such techniques are known in the art and, for this reason are not further elaborated on herein. This screening step may include the extraction and screening of novel enzymes, leading to the recovery, isolation and ultimately modification of novel individual genes as well as linked genes comprising novel gene pathways and to the manufacture and commercialisation of related products.

Initial testing may concentrate on properties of enzymes, using procedures which may include one or more of the following: collection of small or large microbial samples; enzyme extraction and purification; screening of large numbers of purified and semi-purified enzymes for activity under particular conditions. Discovery of an enzyme of interest will provoke investigation of the organism producing it. This may be followed by isolation of the organism and its use to produce the enzyme of interest in commercial quantities under controlled conditions. The DNA of organisms in the bioproducts, particularly those associated with enzymes of interest, may be investigated using techniques such as: creation of gene expression libraries; creation of libraries of multi-gene pathways responsible for the production of small molecules; screening of large numbers of genes and their variants. Such techniques, which may involve storage and manipulation of information generated from screening activities, can lead to discovery of the gene sequence (or gene pathway) leading to the production of an enzyme of interest. This may enable, in suitable cases, insertion of a selected recombinant gene or pathway into host organisms, enabling more efficient manufacture of the desired enzyme (gene expression product) or variants of it improved by genetic recombination.

Enzymes (proteins) isolated in accordance with the present invention may have a multitude of uses, each of which may be identified using an appropriate known screening method. Biological activities may include pharmacological activity against human or animal diseases, or toxicity to insect pests (of humans, animals or plants), or to bacterial or fungal pests of plants. Particular thermostable enzymes have previously been shown to be useful as proteases and lipases—see U.S. Pat. No. 5,714,373 which discloses that such “protease enzymes are useful in a variety of industrial applications including, but not limited to, detergent-based activity, depilating hides, deproteinization of rubber, haze removal in brewing, fish processing, meat tenderization, baking, silver recovery in photographic applications, and removal of protein from xanthum gum fermentation. In the detergent industry, for example, proteases may used to complement the activity of the detergent at temperatures which ordinarily do not support enzyme activity.” Similarly, the US patent discloses that: “Lipases are also useful in industrial applications, for example, in the detergent industry, in fat modification, fat emulsions, cocoa butter, flavoring of milk and cheese products, and in generating solutions of organic acids via esterification in organic media. In addition, lipases have many different applications in the chemical industry. For example, lipases are useful for kinetic resolution of phenylcyclohexanone oxime esters, resolution of racemic acids and alcohols, transesterification of oils, etc.” It is anticipated that the process of the present invention may make it possible to obtain thermostable proteases and lipases useful in such processes. Similar uses for thermostable proteins are proposed in U.S. Pat. No. 5,242,817.

Other important uses for thermostable enzymes are as DNA polymerases. In the Polymerase Chain Reaction (PCR) referred to in the preamble hereof, the thermostable enzyme Taq polymerase is used to amplify traces of nucleic acid sequences, and has many powerful uses, for example in forensic applications in proving or disproving the presence of a suspect at the scene of a crime. Other enzymes have similar uses (see for example U.S. Pat. No. 53,227,850). Similar thermostable DNA polymerases may be produced by the process of the present invention.

The complete disclosures of these US patents, which show various ways of using thermostable enzymes such as may be obtained by the process of the present invention, are incorporated herein by reference.

1. A method of producing bioproducts which includes the steps of:
(a) establishing an environment, under controlled conditions, wherein microorganisms are used to oxidise a slurry containing metal sulphide minerals, and
(b) separating and recovering bioproducts from the slurry.

2. A method according to claim 1 wherein the said environment is established in at least one reactor vessel.

3. A method according to claim 2 wherein the bioproducts which are recovered are microorganisms or their metabolites, or both in admixture.

4. A method according to claim 2 or 3 which includes the step of screening recovered bioproducts for desired properties.

5. A method according to claim 2, 3 or 4 wherein the slurry is supplied to the reactor vessel in a continuous stream.

6. A method according to any one of claims 2 to 5 wherein the metal sulphide minerals are selected from pyrite, arsenopyrite, pentlandite, enargite, chalcopyrite, sphalerite, cinnabar, covelite, and bornite.

7. A method according to any one of claims 2 to 6 which includes the step of adding one or more dissolved metals in a soluble form to the reactor vessel.

8. A method according to any one of claims 2 to 7 which includes the step of introducing small amounts of organic compounds into the reactor vessel to provide greater selectivity for specific microorganisms with a growth requirement which is met by these organic compounds.

9. A method according to any one of claims 2 to 8 which includes the step of adding one or more inorganic nutrients to the slurry in the reactor vessel to optimise the growth of selected microorganisms in the slurry.

10. A method according to claim 9 wherein the inorganic nutrients are selected from nitrogen, phosphorous and potassium.

11. A method according to any one of claims 2 to 10 which includes the step of applying specific mutagenic pressure to the microorganisms in the reactor vessel to promote adaptation of the microorganisms to particular conditions.

12. A method according to claim 11 wherein the mutagenic pressure is selected from exposure of some cells to ultraviolet or gamma radiation, and the introduction of appropriate concentrations of mutagenic chemicals into the slurry.

13. A method according to any one of claims 2 to 12 which includes the step of controlling the mineral solids concentration of the slurry in the reactor vessel to facilitate the selection of microorganisms with associated physiological functions capable of withstanding high shear forces associated with a high solids loading.

14. A method according to any one of claims 2 to 13 wherein the reactor vessel includes an impeller for agitating the slurry and the method includes the step of controlling the impeller speed, or the degree of agitation, in order to select for microorganisms with associated physiological functions capable of withstanding high shear forces associated with high impeller tip speeds.

15. A method according to any one of claims 2 to 14 which includes the step of controlling the hydraulic retention time of the reactor vessel to select for microorganisms with a specific cell growth rate.

16. A method according to any one of claims 2 to 15 which includes the step of sparging the slurry in the reactor vessel with air.

17. A method according to claim 16 wherein the air is supplemented with additional carbon dioxide thereby resulting in stable and elevated dissolved carbon dioxide concentrations.

18. A method according to claim 16 wherein the air is supplemented with additional oxygen thereby resulting in stable and elevated dissolved oxygen concentrations.

19. A method according to any one of claims 2 to 15 which includes the step of feeding an oxygen-enriched gas to the slurry.

20. A method according to claim 19 wherein the feed gas which is supplied to the slurry contains in excess of 85% oxygen by volume.

21. A method according to any one of claims 2 to 20 which includes the step of maintaining the dissolved oxygen concentration in the slurry within the desired range.

22. A method according to claim 21 wherein the dissolved oxygen concentration is maintained in the range of from 0.2×10⁻³ kg/m³ to 10×10⁻³ kg/m³.

23. A method according to any one of claims 2 to 22 which includes the steps of determining the dissolved oxygen concentration in the slurry and, in response thereto, of controlling at least one of the following: the oxygen content of the feed gas, the rate of supply of the feed gas to the slurry, and the rate of feed of the slurry to the reactor vessel.

24. A method according to any one of claims 2 to 23 which includes the step of controlling the carbon content of the slurry by one or more of the following: the addition of carbon dioxide feed gas to the slurry, and the addition of other carbonaceous material to the slurry.

25. A method according to claim 24 which includes the step of controlling the carbon dioxide content of the feed gas to the slurry in the range of from 0.5% to 5% by volume.

26. A method according to claim 24 where the carbon dioxide content of the feed gas is from 1% to 1.5% by volume.

27. A method according to any one of claims 2 to 26 wherein the temperature of the slurry in the reactor vessel is maintained at a value greater than 60° C.

28. A method according to any one of claims 2 to 27 wherein the temperature of the slurry in the reactor vessel is maintained at a value less than 85° C.

29. A method according to any one of claims 2 to 28 which includes the step of initiating microorganisms growth in the reactor by introducing a suitable microorganism inoculum into the slurry.

30. A method according to claim 29 wherein the inoculum is obtained from a source selected from liquid or solid samples from sulphur-containing coal dumps, sulphur-containing volcanic thermal areas and sulphur-containing inland thermal hot springs.

31. A method according to any one of claims 2 to 28 wherein the environment is maintained at a temperature of up to 45° C. and the slurry is oxidised using mesophile microorganisms selected from the following genus groups:
Acidithiobacillus (formerly Thiobacillus); Leptospirillum; Ferromicrobium; and Acidiphilium.

32. A method according to any one of claims 2 to 28 wherein the environment is maintained at a temperature of from 45° C. to 60° C. and the slurry is oxidised using moderate thermophile microorganisms selected from the following genus groups:
Acidithiobacillus (formerly Thiobacillus); Acidimicrobium; Sulfobacillus; Ferroplasma (Ferriplasma); and Alicyclobacillus.

33. A method according to any one of claims 2 to 28 wherein the environment is maintained at a temperature in the range of from 60° C. to 85° C. and the slurry is oxidised using thermophilic microorganisms selected from the following genus groups:
Acidothermus; Sulfolobus; Metallosphaera; Acidianus; Ferroplasma (Ferriplasma); Thermoplasma; and Picrophilus.

34. A method of producing bioproducts which includes the steps of:
(a) culturing microorganisms which are capable of oxidising mineral sulphides contained in a slurry at a temperature in excess of 40° C.,
(b) controlling the dissolved oxygen concentration in the slurry within a predetermined range, and
(c) extracting bioproducts from the slurry.

35. A method according to claim 34 wherein the said predetermined range is from 0.2×10⁻³ kg/m³ to 10×10⁻³ kg/m³ with the concentration value being determined to optimise the growth rate of the microorganisms.

36. A method according to claim 34 or 35 wherein the oxygen is supplied to the slurry in the form of oxygen-enriched gas or substantially pure oxygen.

37. A method according to any one of claims 34 to 36 wherein the said operating temperature is in excess of 60° C.

38. A method according to claim 37 wherein the said operating temperature is in the range of from 60° C. to 85° C.

39. A method according to any one of claims 31 to 35 which includes the step of varying one or more of the following physical parameters relating to the slurry: the dissolved carbon dioxide concentration in the slurry, the temperature of the slurry, the pH of the slurry, the supply of organic nutrients to the slurry, the exposure of the slurry to mutagenic factors, the mineral solids concentration in the slurry, the hydraulic retention time of the slurry in a reactor vessel, and the imposition of the high shear forces on the slurry.

40. A method of producing bioproducts which includes the steps of:
(a) establishing an environment wherein microorganisms oxidise a slurry containing metal sulphide minerals,
(b) supplying a feed gas containing in excess of 21% oxygen by volume to the slurry, and
(c) extracting bioproducts from the slurry.

41. A method according to claim 40 wherein the feed gas contains in excess of 85% oxygen by volume.

42. A method according to claim 40 or 41 which is carried out at a temperature in excess of 60° C.

43. A method according to claim 40, 41 or 42 wherein the bioproducts are proteins and microorganisms.

44. A method according to any one of claims 40 to 43 wherein the extracted bioproducts are investigated using techniques selected from creation of gene expression libraries; creation of libraries of multi-gene pathways responsible for the production of small molecules; screening of large number of genes and their variants.
